(19) United States
(12) Patent Application Publication
(10) Pub. No.: US 2025/0286432 A1
(43) Pub. Date: Sep. 11, 2025

(54) MAGNETIC LEVITATION DEVICE AND AN ELECTROMAGNETIC ROTARY DRIVE

(71) Applicant: Levitronix GmbH, Zürich (CH)

(72) Inventor: Daniel STEINERT, Bülach (CH)

(21) Appl. No.: 19/048,664

(22) Filed: Feb. 7, 2025

(30) Foreign Application Priority Data
Mar. 5, 2024 (EP) 24161376.9

(51) Int. Cl. H02K 7/09; H02K 1/02; H02K 1/12

(52) U.S. Cl. CPC H02K 7/09; H02K 1/12; H02K 1/02

(57) ABSTRACT

A magnetic levitation includes a stator with a cup-shaped recess, which is arranged at an axial end of the stator and into which a rotor can be inserted, The wherein the stator has coil cores, each of which has a longitudinal leg and a pole piece. Each longitudinal leg extends from a first end in an axial direction to a second end, and a contact surface is arranged at the second end. Each pole piece extends from the contact surface in a radial direction to an end face. The radial direction is perpendicular to the axial direction, and the end face is arranged around the cup-shaped recess. A concentrated winding is arranged at each longitudinal leg, and surrounds the respective longitudinal leg. The longitudinal legs and the pole pieces are made of different materials.

### CROSS REFERENCE TO RELATED APPLICATIONS

This Applicant claims priority to European Application Number EP 24161376.9 filed Mar. 5, 2024, the contents of which are herein incorporated by reference.

### BACKGROUND

### Technical Field

The disclosure relates to a magnetic levitation device and to an electromagnetic rotary drive with such a magnetic levitation device.

### Background Information

Magnetic bearing devices for contactless magnetic bearing of a rotor generally have the advantage that they do not require mechanical bearings for the rotor. The rotor is supported or stabilized by magnetic forces which are generated by a stator of the magnetic bearing device. Due to the absence of mechanical bearings, such magnetic bearing devices are in particular suitable for pumping, mixing, centrifuging or stirring devices, with which very sensitive substances are conveyed, for example blood pumps, or on which very high demands are made with respect to purity, for example in the pharmaceutical industry or in the biotechnological industry, or with which abrasive or aggressive substances are conveyed, which would very quickly destroy mechanical bearings, for example pumps or mixers for slurry, sulfuric acid, phosphoric acid or other chemicals in the semiconductor industry.

In the biotechnology industry, such magnetic bearing devices are used, for example, in connection with bioreactors, e.g. in centrifugal pumps for conveying the fluids into or out of the bioreactor, or in mixing devices which mix the fluids in the bioreactor. In the semiconductor industry, such magnetic bearing devices are not only used for conveying aggressive or abrasive substances, but also, for example, for rotation devices with which wafers are rotated.

It is also known to use magnetic bearing devices for viscometers.

### SUMMARY

It has been determined that the temple construction is an advantageous magnetic bearing device design, to which the present disclosure also relates.

The characteristic feature of the temple construction is that the stator of the magnetic bearing device has a plurality of coil cores, each of which comprises a longitudinal leg extending from a first end in an axial direction to a second end. Here, the axial direction refers to that direction which is defined by the desired axis of rotation of the rotor, which is supported by the magnetic bearing device. The desired axis of rotation is that axis of rotation about which the rotor rotates in the operating state when it is in a centered and non-tilted position with respect to the stator. Each coil core comprises, in addition to the longitudinal leg, a transverse leg, also called a pole piece, which is arranged in each case at the second end of the longitudinal leg, and which extends in the radial direction—usually towards inside—, wherein the radial direction is perpendicular to the axial direction. Thus, the transverse leg extends substantially at a right angle to the longitudinal leg. The coil cores each have the shape of an L, wherein the transverse legs form the short legs of the L. The rotor to be supported is then arranged between the transverse legs.

The plurality of the longitudinal legs which extend in the axial direction, and which are reminiscent of the columns of a temple has given this construction its name.

In one design, the stator of the magnetic bearing device has, for example, six coil cores which are arranged circularly and equidistantly around a cup-shaped recess into which the rotor can be inserted. The first ends of the longitudinal legs are usually connected in the circumferential direction by a back iron, which serves to conduct the magnetic flux. The rotor to be supported comprises a magnetically effective core, for example a permanent magnetic disk or a permanent magnetic ring, which is arranged between the radially inner ends of the transverse legs, and which rotates about the axial direction in the operating state, wherein the rotor is magnetically supported without contact with respect to the stator.

For such magnetic bearing devices, it is not necessarily the case that the magnetically effective core of the rotor must be designed in a permanent magnetic manner. There are also known such designs in which the magnetically effective core of the rotor is designed in a permanent magnetic-free manner, i.e., without permanent magnets. Then, the magnetically effective core of the rotor is, for example, designed in a ferromagnetic manner and is made, for example, of iron, nickel-iron, cobalt-iron, silicon iron, mu-metal, or another ferromagnetic material.

Furthermore, designs are possible in which the magnetically effective core of the rotor comprises both ferromagnetic materials and permanent magnetic materials. For example, permanent magnets can be placed or inserted into a ferromagnetic base body. Such designs are advantageous, for example, if one wishes to reduce the costs of large rotors by saving permanent magnetic material.

The longitudinal legs carry windings to generate the electromagnetic fields necessary for the contactless magnetic bearing of the rotor. For example, the windings are designed such that one concentrated winding is wound around each longitudinal leg, i.e., the coil axis of each concentrated winding extends in each case in the axial direction. Here, it is typical for the temple construction that the coil axes of the concentrated windings run in the axial direction and that the concentrated windings are not arranged in the radial plane in which the rotor or the magnetically effective core of the rotor is supported in the operating state.

Designs are possible in which exactly one concentrated winding is arranged on each longitudinal leg. In other designs, several, for example exactly two, concentrated windings are provided on each longitudinal leg. Designs are also possible in which windings are provided that are wound around two longitudinal legs that are adjacent in the circumferential direction, so that these two adjacent longitudinal legs are both located in the interior space of the concentrated winding.

The coil cores of the magnetic bearing devices known from the state of the art are usually designed in sheet metal. This means that several sheet metals in the form of the coil cores are stacked in circumferential direction, insulated from each other. The sheet metal design of the coil cores prevents eddy currents for magnetic fields that run in the direction of the sheet metals, i.e. fields that follow the longitudinal leg in the axial direction and the transverse leg in the radial direction.

For magnetic fields that emerge laterally, i.e. in the circumferential direction, from the sheet metals of the longitudinal leg and the transverse leg, the insulation of the sheet metals is ineffective and thus eddy currents still occur, since these magnetic fields pass orthogonally through the sheet metals.

Particularly with magnetic bearing devices that have a large magnetic gap, wherein the magnetic gap is defined as the distance between the end face of the pole piece and the magnetically effective core of the rotor in the radial direction, orthogonal field components cannot be neglected and generate significant eddy current losses.

In the context of this application, a large magnetic gap means a magnetic gap that is greater than 1% of the diameter of the magnetically effective core in the radial direction. In some cases, the magnetic gap may be greater than or equal to 5% of the diameter of the magnetically effective core in the radial direction.

It is an object of the disclosure to propose a magnetic levitation device for contactless magnetic levitation of a rotor with a disk-shaped or ring-shaped magnetically effective core, which has lower eddy current losses than the previous state of the art.

Furthermore, it is an object of the disclosure to propose an electromagnetic rotary drive with such a magnetic levitation device.

The subject matter of the disclosure meeting this object is characterized by the features disclosed herein.

According to the disclosure, a magnetic levitation device is thus proposed for contactless magnetic levitation of a rotor, which has a disk-shaped or ring-shaped magnetically effective core, wherein the magnetic levitation device has a stator with a cup-shaped recess, which is arranged at an axial end of the stator and into which the rotor can be inserted, wherein the stator has a plurality of coil cores, each of which has a longitudinal leg and a pole piece, wherein each longitudinal leg extends from a first end in an axial direction to a second end, wherein a contact surface is arranged at the second end, wherein each pole piece extends from the contact surface at least partially in a radial direction to an end face, wherein the radial direction is perpendicular to the axial direction, wherein the end face is arranged around the cup-shaped recess, and wherein at least one concentrated winding is arranged at each longitudinal leg, which surrounds the respective longitudinal leg, wherein the longitudinal legs are made of a first material and the pole pieces are made of a second material, and wherein the first material and the second material are different.

The pole piece comprised by the magnetic levitation device according to the disclosure substantially corresponds to the transverse leg of a magnetic bearing device or electromagnetic rotary drive in the temple arrangement (also known as a temple motor) known from the state of the art.

Due to the two-part embodiment of the coil core, wherein the pole piece and the longitudinal leg consist of different materials, the ideal material can be selected for both in order to reduce eddy current losses. By the selection of different materials, it is possible to reduce the eddy current losses where they are particularly high. This is usually the case at the pole pieces.

This means that the selection of an ideal material for the pole piece leads to a reduction in eddy current losses for the entire coil core. The embodiment with different materials thus offers a very flexible adaptation of the coil cores to the corresponding requirements of the magnetic levitation device.

According to a preferred embodiment, each longitudinal leg is made of elements in sheet metal, wherein the elements are stacked in the circumferential direction of the stator. The sheet metal embodiment ensures a reduction in eddy current losses in the longitudinal leg. Here, the stator determines the circumferential direction, and a plurality of coil cores are arranged along this direction in a ring-shaped manner.

Particularly with such a sheet metal embodiment, the selection of different materials for the longitudinal leg and the pole piece is advantageous in order to reduce eddy current losses where they are particularly large. Most eddy current losses occur at the pole pieces, since the distance between adjacent pole pieces is particularly small, in particular at their ends facing the cup-shaped recess. The reason for this is that the eddy current losses mainly result from orthogonal fields, i.e. fields that penetrate the elements orthogonally. Such orthogonal fields have the possibility of flowing in the circumferential direction from the pole piece of a first coil core to the pole piece of a second, adjacent coil core, in particular with small distances between the pole pieces.

Thus, by a suitable selection of the second material that does not correspond to the material of the sheet metal elements of the longitudinal leg, an effective reduction of eddy current losses can be achieved where they occur most strongly.

It is also possible that the longitudinal leg is made of a solid material.

In the process, it is preferred that the first material is an electrical sheet metal. According to the general definition, an electrical sheet metal is understood to be a soft magnetic material for magnetic cores. Materials which have a low coercive field strength are usually called soft magnetic materials. The coercive field strength is that magnetic field strength which is required to demagnetize a material. Within the framework of this application, a soft magnetic material is understood to be a material, which has a coercive field strength, more precisely a coercive field strength of the magnetic polarization, which amounts to less than 2,000 A/m.

There also exists the possibility of using mu-metal for the first material. If the longitudinal leg is made of a solid material, metals or metal compounds such as FeSi are preferred. It is also possible to design the pole pieces in sheet metal.

Furthermore, it is preferred that the second material is a powder composite material, in particular a soft magnetic powder composite material. Here, these materials, known in English as “Soft Magnetic Composite (SMC)”, can be high-purity iron powders with a special surface coating. In this case, the special surface coating is electrically insulating. SMCs are primarily known for their use in conducting high-frequency magnetic fields (frequency >>1 kHz). However, it is not yet common to use them at lower frequencies. A lower frequency refers to frequencies greater than 65 Hz. Eddy current losses are only favored from a frequency of the magnetic field greater than 65 Hz.

Further advantages of the soft magnetic powder composite materials are that they have a very good ability to conduct flux in three dimensions and have a high electrical resistance and high permeability and have thus practically no eddy current losses. It is precisely the ability to conduct flux in three dimensions without generating high eddy current losses that ensures that soft magnetic powder composite materials are used as the second material.

Since soft magnetic powder composite materials have comparatively high hysteresis losses, soft magnetic powder composite material is only used to manufacture the pole pieces in preferred embodiments. In doing so, a balance between the reduction of eddy current losses at the location where they are particularly large and the undesired influence of the powder composite material on the magnetic circuit is provided. A further reason for using powder composite material only for the pole piece is that it is basically a brittle, sintered material with an unknown ageing process, whereby the service life of the pole piece and thus of the magnetic levitation device can be reduced.

Of course, it is also possible to manufacture the longitudinal legs from a soft magnetic powder composite material, while the pole pieces are made from a different material.

The attachment of a pole piece to a longitudinal leg can be carried out using several possible joining methods. These comprise, inter alia, a force-locking joining method, such as clamping or crimping, a form-locking joining method, such as screwing or plugging, or a material-locking connection, such as gluing. It is also possible to make the connection between the longitudinal leg and the pole piece via tongue and groove joints and/or bungs, such as pins, tines or dovetail joints. In a preferred embodiment, the material-locking joining method is realized by gluing. One advantage here is that the glue is in the same strength range as the powder composite material. In comparison to clamps or screws, this has the advantage that no stress increases occur in the powder composite material. On the other hand, the force-locking and form-locking joining methods have the advantage that they have a low susceptibility to defects caused by ageing or errors during gluing, for example.

In a preferred embodiment, the contact surface is designed in a planar manner and arranged at a surface of the longitudinal leg which stands perpendicular on the radial direction. Here, it is particularly preferred that the contact surface is located near the second end of the longitudinal leg.

In another preferred embodiment, the contact surface is arranged at a surface of the longitudinal leg which stands perpendicular on the axial direction. Particularly preferably, the contact surface is arranged here at the second end of the longitudinal leg.

According to another preferred embodiment, the contact surface is designed in an angled manner. In this context, angled can mean that the contact surface is designed in the shape of an “L” and that one area of the contact surface extends in the axial direction and one area in the radial direction.

With regard to the material-locking joining method, this embodiment has a further advantage. Due to the angled embodiment of the contact surface, the available gluing surface is increased compared to the non-angled embodiment of the contact surface.

Furthermore, the tensile load caused by magnetic forces on the pole pieces is reduced by this embodiment.

According to a further preferred embodiment, the contact surface has two partial surfaces, wherein the first partial surface stands perpendicular on the axial direction and the second partial surface stands perpendicular on the radial direction.

In a preferred embodiment, the pole piece is designed in an angled manner, wherein the pole piece comprises two partial pieces, one of which extends in the radial direction and the other in the axial direction.

In a preferred embodiment, the coil core has a rounding at an axially upper end, which redirects the coil core from the axial direction to the radial direction. For example, in the case of an L-shaped coil core, wherein the long part of the “L” is formed by the longitudinal leg and the short part of the “L” is formed by the pole piece, the edge of the pole piece that is radially outward, when viewed from the cup-shaped recess, and extends in the radial plane in the circumferential direction is designed in a rounded manner. This embodiment has the advantage that it has lower eddy current losses and is also easier to realize with regard to construction. This embodiment can be realized with all embodiments of the contact surface, the end face and the pole pieces or longitudinal legs.

In a preferred embodiment, the end face of the pole piece is designed as a curved surface. It is particularly preferred that the curvature of the end face is designed coaxially to the cup-shaped recess. In other words, the end face of the pole piece is a segment of a cylinder surface, wherein the central axis of this cylinder coincides with the central axis of the cup-shaped recess and its radius is larger than that of the cup-shaped recess, so that the end face does not protrude into the cup-shaped recess.

In a preferred embodiment, the end face is designed to be wider with respect to the circumferential direction than the maximum extension of the contact surface in the circumferential direction. This means that one of the two edges of the end face, which extend in the circumferential direction, is longer than one of the edges of the contact surface, which extend in the circumferential direction. If the end face is designed as a curved surface, the length of one of the circular arcs of the segment of the cylinder surface in the radial plane is greater than the length of one of the edges of the contact surface which extend in the circumferential direction.

This widening of the end face in the circumferential direction has the advantage that it favors the magnetic functionality. For example, the passive rigidity and the active levitation forces can be improved.

According to a particularly preferred embodiment, the stator of the magnetic levitation device is designed for generating a torque with which the rotor can be driven magnetically without contact for rotation about the axial direction.

Here, the stator is designed as a bearing and drive stator, which is both the stator of the electric drive and the stator of the magnetic levitation. The electric windings of the stator can be used to generate a magnetic rotating field, which on the one hand exerts a torque on the rotor, which causes its rotation about a desired axis of rotation, and on the other hand exerts an arbitrarily adjustable transverse force on the rotor, so that its radial position can be actively controlled or regulated.

Particularly with regard to the embodiment in which the magnetic levitation device is designed to generate a torque, the embodiment with the wider end face is advantageous, as this favors the magnetic functionality. For example, an increased torque can be generated, or the passive rigidity or the active levitation forces can be improved.

Furthermore, an electromagnetic rotary drive which is designed as a temple motor is proposed by the disclosure wherein the electromagnetic rotary drive comprises a magnetic levitation device according to the disclosure as well as a rotor with a disk-shaped or ring-shaped magnetically effective core, wherein the rotor can be inserted into the cup-shaped recess, and wherein the rotor is designed as the rotor of the electromagnetic rotary drive.

Such electromagnetic rotary drives are also known as bearingless motors. The term bearingless motor refers to an electromagnetic rotary drive in which the rotor is levitated completely magnetically with respect to the stator, wherein no separate magnetic bearings are provided.

Further advantageous measures and embodiments of the disclosure are apparent from the dependent claims.

### BRIEF DESCRIPTION OF THE DRAWINGS

In the following, the disclosure will be explained in more detail with reference to embodiments and with reference to the drawing. In the drawing show:

FIG. 1 is a perspective view of an embodiment of a magnetic levitation device according to the disclosure,

FIG. 2 is a perspective view of a single coil core of the magnetic levitation device from FIG. 1, as well as a plan view of it,

FIGS. 3A-3D are schematic sectional views of several embodiments of a coil core,

FIG. 4 is a perspective view of a first variant of the embodiment of a coil core from FIG. 3B, as well as the plan view of this,

FIG. 5 is a perspective view of a second variant of the embodiment of a coil core from FIG. 3B, as well as the plan view of this,

FIG. 6 is a schematic sectional view of a further embodiment of a coil core,

FIG. 7 is a perspective view of a variant of the embodiment of a coil core from FIG. 6, as well as the plan view of this,

FIG. 8 is a schematic sectional view of a section of an embodiment of a coil core to illustrate the acting tensile forces,

FIG. 9 is a perspective view of a further embodiment of a coil core,

FIG. 10 is a perspective view of a further embodiment of a magnetic levitation device according to the disclosure,

FIG. 11 is a schematic sectional view for an embodiment of a stator housing,

FIG. 12 is a schematic sectional view of a coil core known from the state of the art, as well as a plan view of it.

### DETAILED DESCRIPTION

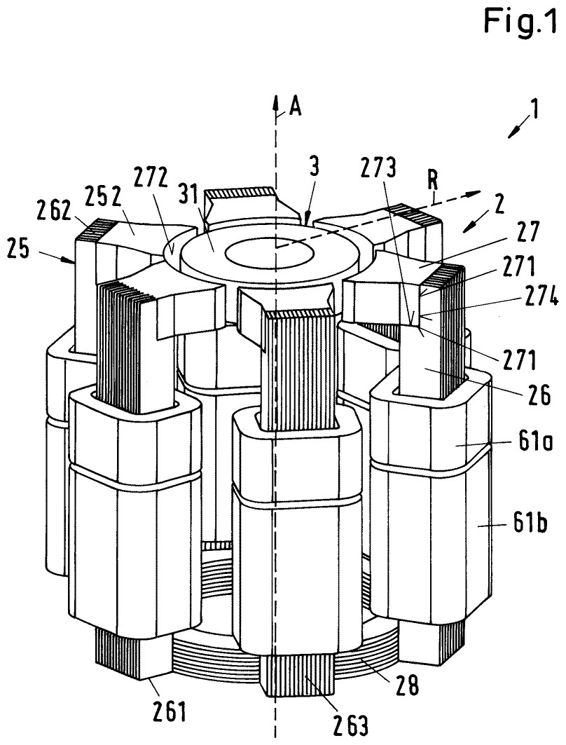

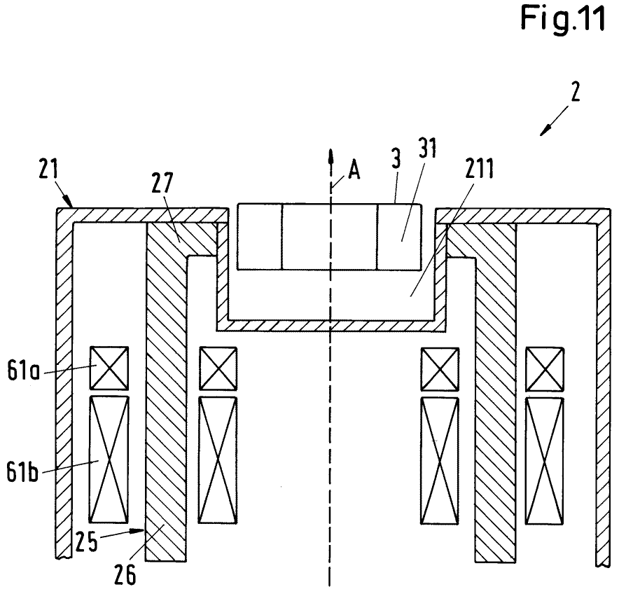

FIG. 1 shows a perspective view of an embodiment of a magnetic levitation device according to the disclosure, which is designated in its entirety by the reference sign 1. The magnetic levitation device 1 is designed for the contactless magnetic levitation of a rotor 3, which comprises a disk-shaped or ring-shaped magnetically effective core 31. The magnetic levitation device 1 is designed according to the temple construction and comprises a stator 2. Normally, the stator 2 comprises a stator housing 21 (FIG. 11), which is not represented in FIG. 1 for reasons of a better overview. A cup-shaped recess 211 (FIG. 11) is provided at one axial end of the stator housing 21 (FIG. 11) into which the rotor 3 can be inserted. The rotor 3 is designed to rotate about a desired axis of rotation. This desired axis of rotation defines an axial direction A. Normally, the central axis of the stator 2, which extends in the axial direction A, coincides with the desired axis of rotation. The desired axis of rotation designates that axis about which the rotor 3 rotates in the operating state when the rotor 3 is in a centered and non-tilted position with respect to the stator 2, as represented in FIG. 1.

The stator 2 has a plurality of coil cores 25-here six coil cores 25-each of which has a longitudinal leg 26 and a pole piece 27. Each longitudinal leg 26 extends from a first end 261 in axial direction A to a second end 262, wherein a contact surface 271 is arranged at the second end 262, from which each pole piece 27 extends at least partially in a radial direction R to an end face 272. Here, the end faces 272 face the rotor 3 and are arranged around it. In other words, the pole pieces 27 of the coil cores 25 are arranged in such a way that the end faces 272 of the pole pieces 27 are arranged around the cup-shaped recess 211 (FIG. 11). The coil cores 25 of the stator 2 are arranged equidistantly on a circular line, so that the end faces 272 surround the magnetically effective core 31 of the rotor 3 when the rotor 3 is inserted into the cup-shaped recess 211 (FIG. 11).

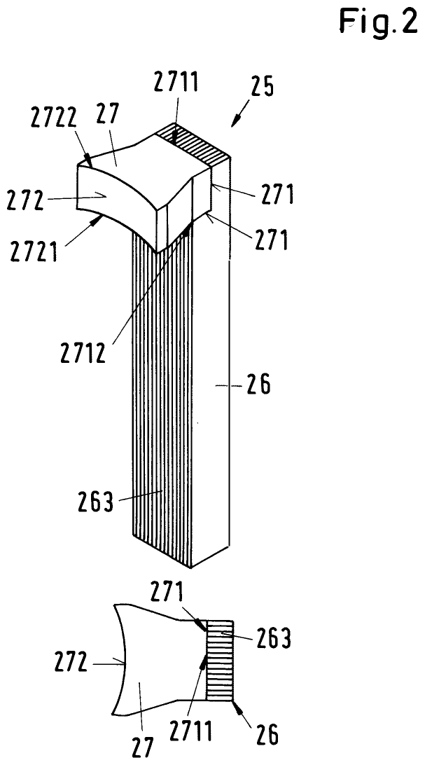

For better understanding, a perspective view of a single coil core 25 of the magnetic bearing device 1 from FIG. 1, as well as a plan view of it is represented in FIG. 2.

At least one concentrated winding 61 is arranged at each longitudinal leg 26, which surrounds the respective longitudinal leg 26. In other embodiments, more than one concentrated winding may also be arranged at the longitudinal legs 26. For example, there are embodiments wherein exactly two concentrated windings are provided in each case on each of the longitudinal legs 26, each of which surrounds the respective longitudinal leg 26, wherein the two windings arranged on the same longitudinal leg 26 are arranged adjacent to each other with respect to the axial direction A.

The concentrated windings 61 serve to generate electromagnetic fields with which the rotor 3 can be levitated magnetically without contact in the cup-shaped recess 211 (FIG. 11).

In this embodiment, the longitudinal legs 26 are made of elements 263 in sheet metal, wherein the elements 263 are stacked in the circumferential direction of the stator 2. The circumferential direction refers to that direction which stands perpendicular on the radial direction R and perpendicular on the axial direction A. Here, the longitudinal legs 26 are made of a first material, in this case an electrical sheet metal, and the pole pieces 27 are made of a second material, wherein this is a powder composite material, preferably a soft magnetic powder composite material. Thus, the first material and the second material are different.

The number of elements 263 in all embodiments and figures is to be understood as purely exemplary. The number may be larger or smaller than represented.

In this embodiment, the contact surface 271 is designed in an angled manner. The contact surface 271 has two partial surfaces, wherein the first partial surface 273 stands perpendicular on the axial direction and the second partial surface 274 stands perpendicular on the radial direction.

In this embodiment, the end face 272 of the pole piece 27 is designed as a curved surface. The curvature of the end face 272 is designed coaxially to the cup-shaped recess 211 (FIG. 11). Here, the end face 272 is designed to be wider with respect to the circumferential direction than the maximum extension of the contact surface 271 in the circumferential direction.

In other words, the end face 272 of the pole piece 27 is a segment of a cylinder surface, wherein the central axis of this cylinder coincides with the central axis of the cup-shaped recess 211 (FIG. 11), in this embodiment the axis of the axial direction A, and whose radius is larger than that of the cup-shaped recess 211 (FIG. 11), so that the end face 272 does not protrude into the cup-shaped recess 211 (FIG. 11).

In a preferred embodiment, the end face 272 is designed to be wider with respect to the circumferential direction than the maximum extension of the contact surface 271 in the circumferential direction. This means that one of the two edges 2721 or 2722 of the end face 272, which extend in the circumferential direction, is longer than one of the edges 2711 or 2712 of the contact surface 271, which extend in the circumferential direction. If the end face 272 is designed as a curved surface, the length of one of the circular arcs 2721 or 2722 of the segment of the cylinder surface in a radial plane E is greater than the length of one of the edges 2711 or 2712 of the contact surface 271, which extend in the circumferential direction. The radial plane is indicated in FIG. 1 by the line of a radial direction R, which stands perpendicular on the axial direction A. The radial plane E is that plane which stands perpendicular on the axial direction A and contains a radial direction R. The radial plane E is that plane in which the magnetically effective core 31 of the rotor 3 is actively magnetically levitated between the end faces 272 in the stator 2 in the operating state. If the rotor 3 is not tilted and is not deflected in axial direction A, the magnetic center plane lies in the radial plane E. The radial plane E defines the x-y plane of a Cartesian coordinate system whose z-axis runs in axial direction A.

The radial position of the magnetically effective core 31 or the rotor 3 refers to the position of the rotor 3 in the radial plane E.

According to a particularly preferred embodiment, the stator 2 is designed in such a way that, in addition to the contactless magnetic levitation of the rotor 3, it can also exert a torque on the rotor 3 or the magnetically effective core 31 of the rotor 3, which drives the rotor 3 to rotate about the desired axis of rotation. This means that in this preferred embodiment, the rotor 3 can be driven for rotation about the axial direction A.

The already mentioned widening of the end face 272 in the circumferential direction has the advantage that it favors the magnetic functionality. For example, the passive rigidity and the active levitation forces can be improved. With regard to the embodiment in which the magnetic levitation device 1 is designed to generate a torque, there is also the advantage that an increased torque can be generated.

In this embodiment, the concentrated windings 61 thus generate electromagnetic rotating fields with which the rotor 3 can be both magnetically levitated without contact with respect to the stator 2 and can also be driven without contact for rotation about the axial direction A.

It is understood that the number of six coil cores 25, although preferred, is only to be understood as an example. Of course, such embodiments are also possible in which the stator 2 has fewer than six, e.g. five or four or three coil cores 25, or such embodiments in which the stator 2 has more than six, e.g. seven or eight or nine coil cores 25 or any larger number of coil cores 25.

The rotor 3 comprises the magnetically effective core 31, which is designed in a ring-shaped or disk-shaped manner. According to the representation in FIG. 1, the magnetically effective core 31 is designed as a ring and defines a magnetic center plane. Alternatively, the magnetically effective core 31 can also be designed as a disk. Normally. in the case of a disk-shaped or ring-shaped magnetically effective core 31, the magnetic center plane is the geometric center plane of the magnetically effective core 31 of the rotor 3, which is perpendicular to the axial direction A. In the operating state, the magnetically effective core 31 is levitated in a radial plane E, which stands perpendicular on the axial direction A.

Since it is sufficient for the understanding of the disclosure, only the magnetically effective core 31 of the rotor 3 is represented in the drawing in FIG. 1. It is understood that the rotor 3 can of course also comprise further components such as jackets or encapsulations, which are preferably made of a plastic, or of a metal or of a metal alloy or of a ceramic or a ceramic material. Furthermore, the rotor 3 can also comprise vanes for mixing, stirring or pumping fluids or other components.

When the rotor 3 is inserted into the cup-shaped recess 211 (FIG. 11), the rotor 3 and in particular the magnetically effective core 31 of the rotor 3 is surrounded by the radially outwardly arranged end faces 272 of the pole pieces 27 of the coil cores 25 of the stator 2. Thus, the pole pieces 27 form a plurality of pronounced stator poles—in this case six stator poles.

When the magnetically effective core 31 of the rotor 3 is in its desired position during operation, the magnetically effective core 31 is centered between the end faces 272 of the pole pieces 27. According to the representation, the concentrated windings 61 are arranged below the radial plane E and are aligned such that their coil axes extend in axial direction A.

All first ends 261 of the longitudinal legs 26-i.e., the lower ends 261 according to the representation (FIG. 1)—are connected to each other by a back iron 28. The back iron 28 is preferably designed in a ring-shaped manner. Such embodiments are possible (see FIG. 1, for example) in which the back iron 28 extends radially inwardly along all first ends 261 of the longitudinal legs 26.

In order to generate the electromagnetic fields required for the magnetic levitation of the rotor 3 and optionally for the generation of a torque on the rotor 3, the longitudinal legs 26 of the coil cores 25 carry the windings designed as concentrated windings 61.

In the operating state, those electromagnetic rotating fields are generated with these concentrated windings 61 with which an arbitrarily adjustable transverse force in the radial direction can be exerted on the rotor 3, so that the radial position of the rotor 3, i.e. its position in the radial plane E perpendicular to the axial direction A, can be actively controlled or regulated. Optionally, a torque is additionally effected on the rotor 3 with these electromagnetic rotating fields.

The “magnetically effective core 31” of the rotor 3 refers to that region of the rotor 3 which magnetically cooperates with the stator 2 for the generation of magnetic levitation forces and optionally for torque generation.

As already mentioned, the magnetically effective core 31 is designed in a ring-shaped manner in this embodiment. Furthermore, the magnetically effective core 31 is designed in a permanent magnetic manner. For this purpose, the magnetically effective core 31 can comprise at least one permanent magnet, but also several permanent magnets, or—as in the embodiment described here—consist entirely of a permanent magnetic material, so that the magnetically effective core 31 is the permanent magnet. For example, the magnetically effective core 31 is magnetized in the radial direction.

Those ferromagnetic or ferrimagnetic materials, which are magnetically hard, that is, which have a high coercive field strength, are typically called permanent magnets. The coercive field strength is that magnetic field strength, which is required to demagnetize a material. Within the framework of this application, a permanent magnet is understood as a component or a material, which has a coercive field strength, more precisely a coercive field strength of the magnetic polarization, which amounts to more than 10,000 A/m.

Such embodiments are also possible in which the magnetically effective core 31 is designed in a permanent magnet-free manner, i.e., without permanent magnets. The rotor 3 is then designed, for example, as a reluctance rotor. Then, the magnetically effective core 31 of the rotor 3 is made of a soft magnetic material, for example. Suitable soft magnetic materials for the magnetically effective core 31 are, for example, ferromagnetic or ferrimagnetic materials, i.e., in particular iron, nickel-iron, cobalt-iron, silicon iron, mu-metal.

Furthermore, embodiments are possible in which the magnetically effective core 31 of the rotor 3 comprises both ferromagnetic materials and permanent magnetic materials. For example, permanent magnets can be placed or inserted into a ferromagnetic base body. Such embodiments are advantageous, for example, if one wishes to reduce the costs of large rotors by saving permanent magnetic material.

Embodiments are also possible in which the rotor is designed according to the principle of a cage rotor.

The stator 2 is free of permanent magnets. Within the framework of this application, the term that the stator 2 is designed “free of permanent magnets” is to be understood to mean that the stator 2 does not comprise any permanent magnets that contribute substantially to the drive field for driving the rotation of the rotor 3 or for generating the magnetic levitation forces for the rotor 3. Thus, the magnetic flux generated by the stator 2 for the drive and the levitation the rotor 3 does not comprise any permanent-magnetically excited flux.

Of course, it is possible that the rotor 3 and/or the stator 2 comprise other magnets or permanent magnets, for example in sensors that serve, for example, to capture the angular position of the rotor, or that otherwise fulfill a purpose that has nothing to do with generating the magnetic flux for the drive and the levitation of the rotor 3.

Thus, the term “free of permanent magnets” refers only to the generation of the magnetic flux for the drive and the levitation of the rotor 3 by the stator 2. In other words, the stator 2 has no permanent magnets that contribute to the magnetic flux by which the rotor 3 is driven and magnetically levitated.

However, it is still possible that the magnetic flux for the drive and the levitation of the rotor 3 comprises a permanent magnetic flux, but this is then only generated by the rotor 3 itself. This would be the case if the rotor 3 itself were to comprise a permanent magnet.

The ring-shaped back iron 28 can be made of a soft magnetic material because it serves as flux conducting element to conduct the magnetic flux.

Suitable soft magnetic materials for the back iron 28 are, for example, ferromagnetic or ferrimagnetic materials, i.e., in particular iron, nickel-iron, cobalt-iron, silicon-iron or mu-metal. In this case, for the stator 2, a design as a stator sheet metal stack is preferred, in which the back iron 28 is designed in sheet metal, i.e., it consists of several thin sheet metal elements, which are stacked parallel to each other in axial direction A. All back iron elements are identically designed, i.e., in this case substantially ring-shaped and also with the same thickness in each case. Thus, the back iron 28 itself is substantially designed in a ring-shaped manner and extends radially inwardly along the first ends 261 of the longitudinal legs 26 in the assembled state.

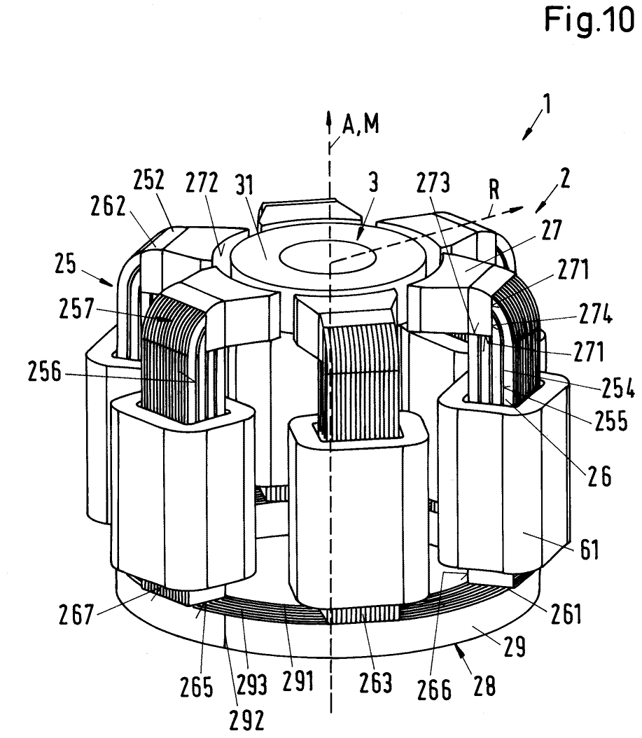

Embodiments in which a so-called tape wound toroidal core is used as the back iron 28 are also conceivable. Such a back iron 28 is represented in the embodiment in FIG. 10. This is a coiled strip made of electrical sheet metal. Preferably, grain-oriented electrical sheet metal is used here. In the state of the art, tape wound toroidal cores are known primarily for use in transformers, transformers, inductors but not for bearing devices and particularly not for electromagnetic rotary drives.

Furthermore, it is possible that the back iron 28 includes pressed and subsequently sintered grains of the aforementioned materials. The metallic grains are preferably embedded in a plastic matrix so that they are at least partially insulated from each other, whereby eddy current losses can be minimized. Thus, soft magnetic composites, which consist of electrically insulated and compressed metal particles are also suitable for the stator. In particular, these soft magnetic composites, which are also designated as SMC (Soft Magnetic Composites), can consist of iron powder particles which are coated with an electrically insulating layer. These SMC are then formed into the desired shape by powder metallurgy processes.

During operation of the magnetic levitation device 1, the magnetically effective core 31 of the rotor 3 cooperates with the stator 2 in such a way that the rotor 3 can be magnetically levitated without contact with respect to the stator 2 and preferably can also be magnetically set in rotation without contact about the axial direction A. In this case, it is particularly advantageous that the same windings 61, with which the magnetic levitation of the rotor 3 is effected, also serve to generate a torque on the rotor 3. Preferably, three degrees of freedom of the rotor 3 can then be actively regulated, namely its position in the radial plane E and its rotation. With respect to its axial deflection from the radial plane E in axial direction A, the magnetically effective core 31 of the rotor 3 is passively magnetically stabilized by reluctance forces, i.e., it cannot be controlled. The magnetically effective core 31 of the rotor 3 is also passively magnetically stabilized with respect to the remaining two degrees of freedom, namely tilting with respect to the radial plane E perpendicular to the desired axis of rotation. By the cooperation of the magnetically effective core 31 with the coil cores 25, the rotor 3 is thus passively magnetically levitated or passively magnetically stabilized in axial direction A and against tilting (a total of three degrees of freedom) and actively magnetically levitated in the radial plane (two degrees of freedom).

As is generally the case, an active magnetic levitation is also referred to in the framework of this application as one which can be actively controlled or regulated, for example by the electromagnetic fields generated by the concentrated windings 61. A passive magnetic levitation or a passive magnetic stabilization is one that cannot be controlled or regulated. The passive magnetic levitation or stabilization is based, for example, on reluctance forces, which bring the rotor 3 back again to its desired position when it is deflected from its desired position, i.e., for example, when it is displaced or deflected in axial direction A or when it is tilted.

In the magnetic levitation device 1, in contrast to classic magnetic bearings, the magnetic levitation—and optionally the generation of a torque acting on the rotor—is realized by electromagnetic rotating fields. For the combined generation of the magnetic levitation forces and a torque for rotating the rotor 3 about the axial direction A, it is possible on the one hand—as shown in FIG. 1—to arrange exactly one concentrated winding 61 at each longitudinal leg 26.

On the other hand, embodiments are also possible in which two different winding systems are provided for the combined generation of the magnetic levitation forces and a torque for rotating the rotor 3. For this purpose, for example, exactly two concentrated windings are arranged in each case at each longitudinal leg 26, which are arranged adjacent to each other with respect to the axial direction A. One of these two windings belongs to the first of the two winding systems and the other to the second of the two winding systems.

In the embodiment represented in FIG. 1 with exactly one concentrated winding 61 at each coil core 25, for example the values for the current required for the levitation and the current required for the generation of the torque determined in each case in the control unit 40 are added or superimposed by calculation—e.g., with the aid of software. Then, the resulting total current is impressed into the respective concentrated winding 61.

If the stator 2 of the magnetic levitation device 1 according to the disclosure is designed for generating a torque, the magnetic levitation device 1 is suitable for an electromagnetic rotary drive, which is designed as a temple motor. It is also possible that the magnetic levitation device 1 according to the disclosure is also suitable for other devices, such as centrifugal pumps, mixing devices for mixing flowable substances, stirring devices, for example for mixing a fluid in a tank, fans or also for devices for supporting and rotating wafers, for example in semiconductor production.

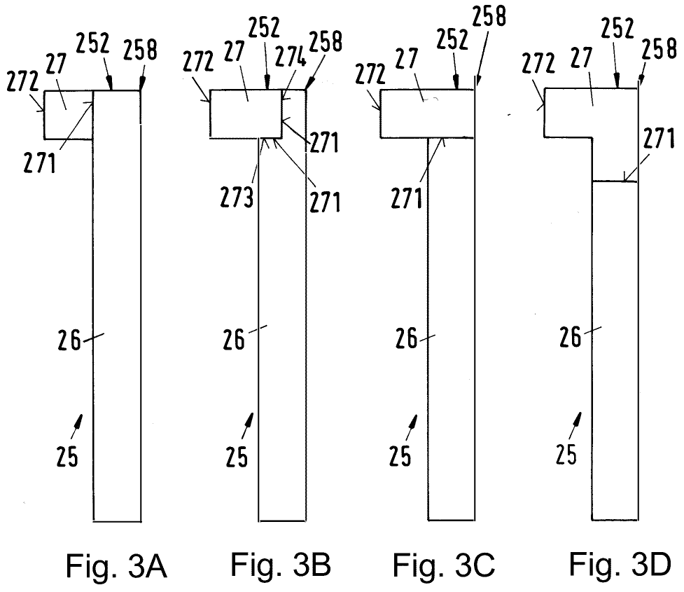

FIG. 3 shows schematic sectional views of several embodiments of a coil core 25.

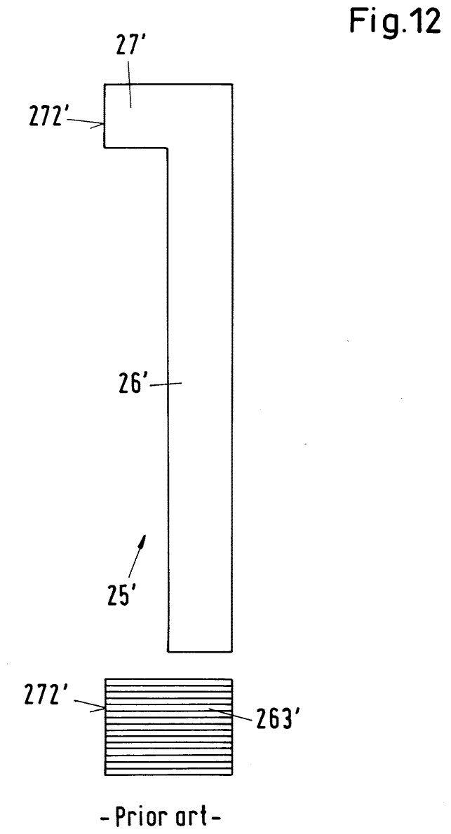

As a comparison, FIG. 12 shows a schematic sectional view of a coil core 25′ known from the state of the art, as well as a plan view of it. Here, the coil core 25′ is designed in one piece and the longitudinal leg 26′ and the pole piece 27′ form a unit. Here, the complete coil core 25′ is made of elements 263′ in sheet metal.

For all embodiments of a coil core 25 illustrated in FIG. 3, the longitudinal leg 26 should preferably be made of elements 263 in sheet metal, wherein the elements 263 are stacked in the circumferential direction of the stator 2, and wherein the longitudinal legs 26 are made of electrical sheet metal and the pole pieces 27 are preferably made of a powder composite material, particularly preferably a soft magnetic powder composite material.

FIG. 3A shows a further embodiment of a coil core 25, in which the contact surface 271 is designed in a planar manner and is arranged at a surface of the longitudinal leg 26 which stands perpendicular on the radial direction. Thus, the pole piece 27 is arranged at a side surface of the longitudinal leg 26. In this variant, the pole piece 27 is located at the upper second end 262 of the longitudinal leg 26.

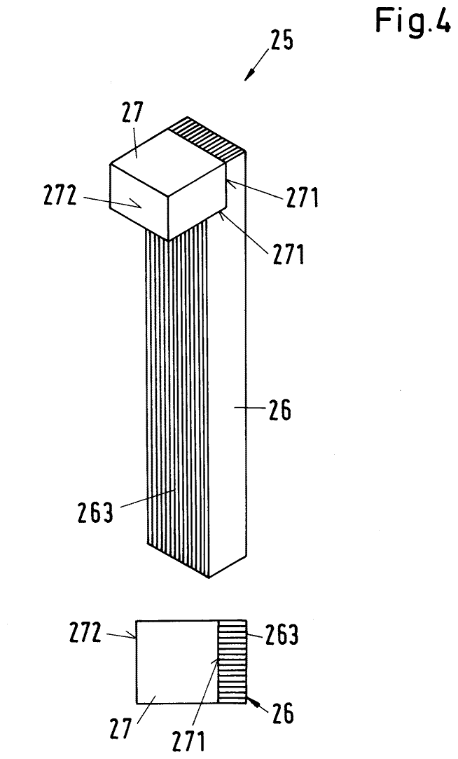

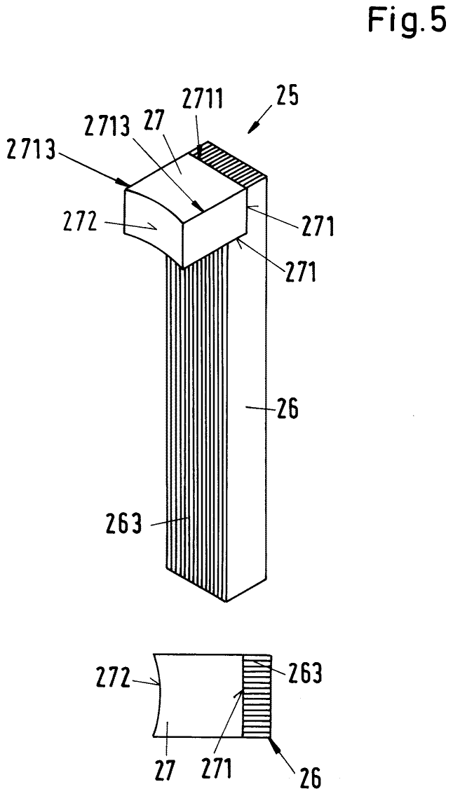

FIG. 3B shows a further embodiment of a coil core 25 in which the contact surface 271 is designed in an angled manner. Thus, the contact surface 271 can be divided into two partial surfaces, wherein the first partial surface 273 stands perpendicular on the axial direction and the second partial surface 274 stands perpendicular on the radial direction. The coil cores 25 from FIG. 1 or FIG. 2 fall into the group of this embodiment. In FIGS. 4 and 5, two further possible variants of this embodiment of a coil core 25 are represented.

FIG. 3C shows a further embodiment of a coil core 25, in which the contact surface 271 is arranged at a surface of the longitudinal leg 26 which stands perpendicular on the axial direction. Thus, the pole piece 27 is arranged at the surface at the upper second end 262 of the longitudinal leg 26.

FIG. 3D shows a further embodiment of a coil core 25, in which the contact surface 271 is again arranged at a surface of the longitudinal leg 26 which stands perpendicular on the axial direction. Thus, the pole piece 27 is arranged at the surface at the upper second end 262 of the longitudinal leg 26. The special feature of this embodiment is that the pole piece 27 is designed in an angled manner. This means that the pole piece 27 comprises two partial pieces, one of which extends in radial direction R and the other in axial direction A. The pole piece 27 thus has the shape of an “L”. The two partial pieces of the pole piece 27 do not necessarily have to have the same edge lengths. It is also possible that one partial piece has a longer edge length than the other. Likewise, the angle between the two partial pieces is not necessarily 90°. Embodiments are also possible in which an angle other than 90° is enclosed by the two partial pieces.

FIG. 4 shows a perspective view of a first variant of the embodiment of a coil core 25 from FIG. 3B (top), as well as the plan view of this from the axial direction (bottom), i.e. according to the representation from above. In this variant, the end face 272 has no curvature, but is designed as a planar surface. In this variant, the longitudinal leg 26 is made of elements 263 in sheet metal, the material of which is preferably electrical sheet metal. The pole piece 27 is made of a powder composite material, preferably a soft magnetic powder composite material.

FIG. 5 shows a perspective view of a second variant of the embodiment of a coil core 25 from FIG. 3B (top), as well as the plan view of this from the axial direction (bottom), i.e. according to the representation from above. In this variant, the end face 272 of the pole piece 27 is designed as a curved surface. The curvature of the end face 272 is designed coaxially with the cup-shaped recess 211 (FIG. 11).

In this variant, the end face 272 is not designed to be wider with respect to the circumferential direction than the maximum extension of the contact surface 271 in the circumferential direction. This means that the edge 2711 encloses an angle of 90° in each case with the two side edges 2713 of the pole piece 27.

Of course, the described variants and embodiments of the coil cores 25 from the FIG. 2-5 can also be combined with each other in any form. This means that it is also possible that in each of the embodiments of a coil core 25 from FIGS. 3A-3D, for example, the end face 272 of the pole piece 27 is designed as a curved surface or, for example, the end face 272 is designed to be wider with respect to the circumferential direction than the maximum extension of the contact surface 271 in the circumferential direction.

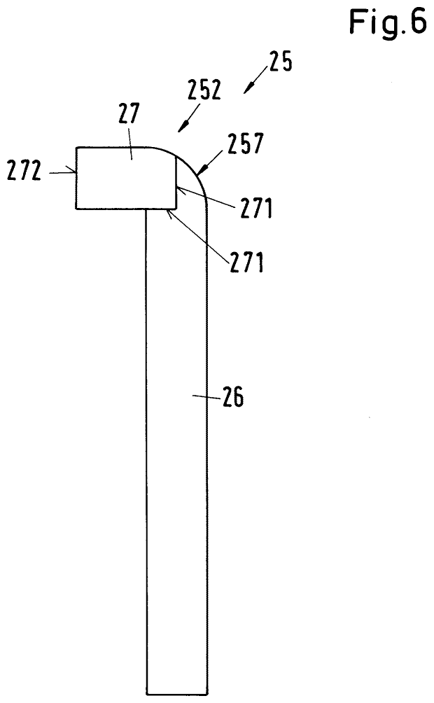

FIG. 6 shows a schematic sectional view of a further embodiment of a coil core 25. Here, the coil core 25 has a rounding at an axially upper end 252, which redirects the coil core 25 from the axial direction A to the radial direction R. In this embodiment, the coil core 25 and the contact surface 271 are designed according to the embodiment analogous to FIG. 3B and the coil core 25 additionally has a rounding 257 of the outer edge 258 of the coil core 25 at the axially upper end of the coil core 252. This means that in this embodiment, in comparison to the embodiments in FIGS. 3A-3D, the radially outer edge 258 at the axially upper end 252 of the coil core 25 is a broken edge or a rounded edge, as represented by the rounding 257. Depending on the embodiment of the coil core 25, the rounding 257 can extend only in areas of the longitudinal leg 26 or only in areas of the pole piece 27 or both in areas of the longitudinal leg 26 and of the pole piece 27.

This embodiment has the advantage that it has lower eddy current losses and is also easier to realize with regard to the construction. Of course, this embodiment can be realized or combined with all other embodiments of the coil cores 25 with regard to the contact surface 271, the end face 272, the pole pieces 27 and the longitudinal legs 26.

With regard to the structure and materials of the longitudinal leg 26 and the pole piece 27, the same advantages as in the embodiments of a coil core 25 explained in FIG. 3 are of importance.

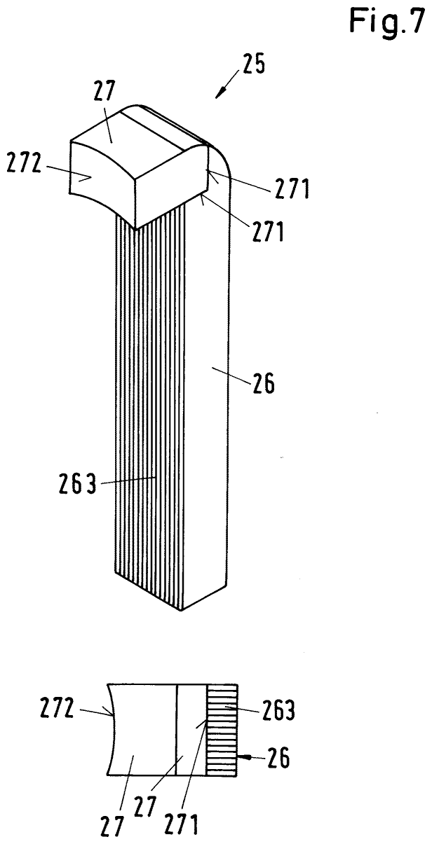

FIG. 7 shows a perspective view of a variant of the embodiment of a coil core 25 from FIG. 6 (top), as well as the plan view of this from axial direction (bottom), i.e. according to the representation from above. In this variant, the end face 272 of the pole piece 27 is designed as a curved surface, wherein the curvature of the end face 272 is designed coaxially with the cup-shaped recess 211 (FIG. 11).

Likewise, variants are also possible in which the end face 272 of the pole piece 27 is designed to be wider with respect to the circumferential direction than the maximum extension of the contact surface 271 in the circumferential direction. Furthermore, the end face 272 can also have no curvature, but simply be a planar surface.

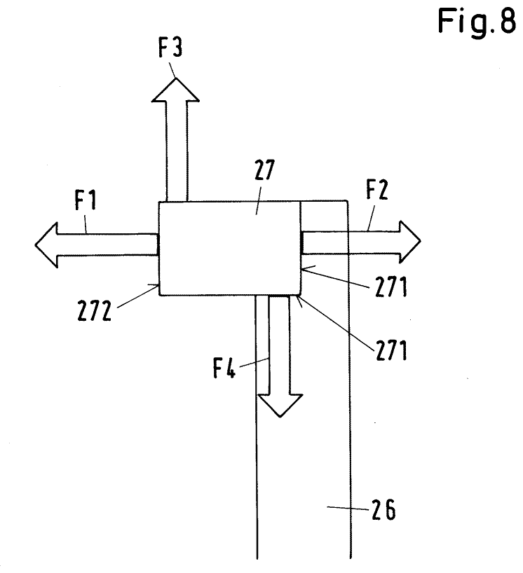

FIG. 8 shows a schematic sectional view of a section of an embodiment of a coil core 25 to illustrate the acting tensile forces. This schematic view serves purely to illustrate the magnetic tensile forces acting on the embodiment of the coil core 25 analogous to FIG. 3B. The four thick arrows with the reference signs F1-F4 symbolize the force vectors acting on the respective surfaces. Since the rotor 3 has a magnetically effective core 31, it is attracted to the coil core 25, more precisely to the pole piece 27. Since this attraction is an interaction, there must be an opposing force of the same magnitude so that the rotor 3 can be levitated in the magnetic levitation device 1. This means that a force also acts on the pole piece 27. The force acting on the pole piece 27 is transmitted via the pole piece 27 to the longitudinal leg 26 and thus also to the contact surface 271. Depending on how the contact surface 271 is designed, different force vectors act on it. In the case of the non-angled contact surfaces 271 (cf. FIGS. 3A and 3C), the forces only act either in axial direction A or in radial direction R, depending on the embodiment. This means that the entire force acting on the contact surface 271 is only transmitted to the longitudinal leg 26 by one force vector. As a result, the requirements for the joining methods in these two examples are very high so that a stable and reliable connection of the pole piece 27 and the longitudinal leg 26 at the contact surface 271 can be created. For this reason, it is advantageous if the contact surface 271 is optimized as represented in FIG. 8 in such a way that it is designed at an angled manner. In this embodiment, the force acting on the contact surface 271 is divided into two forces F2, F4. This means that both a force vector in axial direction A and a force vector in radial direction R act on the longitudinal leg 26. This has the advantage that only very low loads due to magnetic forces act on the contact surface 271, and thereby the susceptibility of the coil cores 25 to defects is reduced.

A further advantage resulting from this is that more cost-effective joining methods can be used for a stable and reliable connection of the pole piece 27 and the longitudinal leg 26. With regard to the material-locking joining method, this embodiment has a further advantage. Due to the angled contact surface 271, the available gluing surface compared to the non-angled embodiments of the contact surface 271 is increased and thus a more stable and more reliable connection between the pole piece 27 and the longitudinal leg 26 is possible.

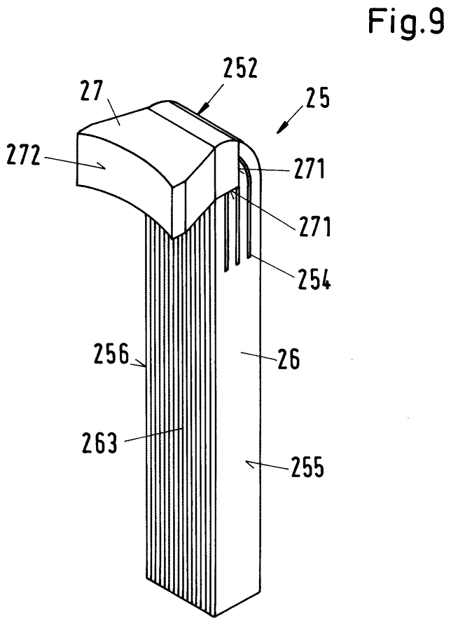

FIG. 9 shows a perspective view of a further embodiment of a coil core 25. Here, the longitudinal leg 26 is again made of elements 263 in sheet metal, wherein the elements 263 are stacked in the circumferential direction of the stator 2, wherein each coil core 25 has a first lateral boundary surface 255 and a second lateral boundary surface 256. The pole piece 27 has a curvature of the end face 272 coaxial with the cup-shaped recess 211 (FIG. 11). With respect to the circumferential direction, the end face 272 is designed to be wider than the maximum extension of the contact surface 271 in the circumferential direction. In this embodiment, the material of the pole piece 27 is a powder composite material, preferably a soft magnetic powder composite material. However, it is also possible that the material of the pole piece 27 can be a different material. The contact surface 271 is designed in an L-shaped and the coil core 25 has a rounding at the axially upper end 252, which redirects the coil core 25 from the axial direction A to the radial direction R. In this embodiment, at least one of the first or the second lateral boundary surfaces 255, 256 has three slots 254. However, other embodiments are also possible in which fewer than three or more than three slots 254 are present. The slots 254 extend not only in the two lateral boundary surfaces 255, 256 but may also be present in the inner elements 263. Embodiments are possible in which a plurality of slots 254 extend through all elements 263.

In this embodiment, the advantage is that the slots 254 are an electrical insulation, whereby they block the path of the eddy currents. Due to this measure, eddy current losses are again significantly reduced.

FIG. 10 shows a perspective view of a further embodiment of a magnetic levitation device 1 according to the disclosure. In the following description of the further embodiment of the magnetic levitation device 1, only the differences to the first embodiment from FIG. 1 are explained in more detail. The explanations of the first embodiment also apply in the same way or analogously to the second embodiment. The same reference signs designate the same features that were explained with reference to the first embodiment, or features equivalent in function.

In this embodiment in FIG. 10, the magnetic levitation device 1 comprises the coil cores 25 represented in FIG. 9. A further difference between this embodiment of a magnetic levitation device 1 and the embodiment from FIG. 1 is that the back iron 28 is designed differently. The back iron 28 is designed in a ring-shaped manner with a metallic strip 29, which extends from a radially inner beginning 291 to a radially outer end 292. The strip 29 forms several strip windings 293, which lie flat against each other with respect to the radial direction R. The longitudinal legs 26 are delimited at the first end 261 by an axial end face 265, against which the back iron 28 rests. The back iron 28 forms a circular ring, the radial width of which is equal to the radial width of the end faces 265 of the longitudinal legs 26. This means that the radially inner beginning 291 is flush in axial direction A with a radially inner surface 266 of the longitudinal leg 26 and the radially outer end 292 is flush in axial direction A with a radially outer surface 267 of the longitudinal leg 26.

Inter alia, this has the advantage that there is more space inside the stator 2, which can be used to install other components in it. In doing so, the stator 2 together with the stator housing 21 (FIG. 11) can be made smaller and more compact, which increases the flexibility of use of the magnetic levitation device 1.

Needless to say, that all embodiments of a coil core 25 shown in the figure description can be combined with their respective characteristics in any form. Likewise, of course, all embodiments of a coil core 25 can be used in the embodiments of a magnetic levitation device 1.

Furthermore, it is possible that all of the shown embodiments of a coil core 25 can be designed in such a way that the space available to the rotor 3 in the magnetic levitation device 1 is increased. This is achieved by a special external shape of the coil cores 25.

In the process, the coil core 25 is divided into an axially lower section and an axially upper section, wherein the lower section and the upper section are arranged adjacent to each other with respect to the axial direction A. The pole piece 27 is arranged at the axially upper section. For each coil core 25, the end face 272 of the pole piece 27 has a first distance in radial direction from the axially lower section of the associated longitudinal leg 26, and a second distance in radial direction from the axially upper section, wherein the second distance is greater than the first distance. This means that each longitudinal leg 26 is designed in such a way that the axially upper section is displaced outwards in radial direction with respect to the axially lower section, so that the space available for the rotor 3 between the end faces 272 is increased without the risk of a direct transfer of the magnetic flux between the longitudinal leg 26 and the magnetically effective core 31 of the rotor 3 existing. Due to the fact that the axially upper sections are offset radially outwards with respect to the radial direction and relative to the axially lower sections, the distance, namely the second distance, between the longitudinal legs 26 and the end faces 272 increases in the area of the axially upper sections. In doing so, the distance between the magnetically effective core 31 of the rotor 3 and the longitudinal legs 26, particularly in the area of the axially upper sections, also increases.

Such coil cores 25 just described are designed analogously to those coil cores illustrated in FIG. 3 in the European patent application EP4084304A1.

FIG. 11 shows a schematic sectional view for an embodiment of a stator housing 21. In the embodiments in FIG. 1 and FIG. 10, this stator housing 21 is not represented for reasons of a better overview. FIG. 11 is only intended to serve as an illustration to show how an encapsulation of the interior of the stator 2 necessary for the operation of the magnetic levitation device 1 looks. For this reason, the other components of the stator 2 are only represented schematically and are to be understood as purely illustrative.

Embodiments of the stator housing 21 are also possible in which the cup-shaped recess 211 merges into a bore that extends centrally along the central axis of the stator 2 in axial direction A through the entire stator housing 21.

During operation of the magnetic levitation device 1 in fields in which, for example, chemically aggressive substances are used, it is important that the interior of the stator 2 is securely encapsulated and thus protected from these substances. To ensure that a rotor 3 can still be used, the stator housing 21 has a cup-shaped recess 211 into which the rotor 3 can be inserted.

What is claimed is:

1. A magnetic levitation device for contactless magnetic levitation of a rotor, which has a disk-shaped or ring-shaped magnetically effective core, comprising:
a stator with a cup-shaped recess arranged at an axial end of the stator and into which the rotor is capable of being inserted, the stator having a plurality of coil cores, each of the plurality of coils having a longitudinal leg and a pole piece, for each of the plurality of coils, the longitudinal leg extends from a first end in an axial direction to a second end, and a contact surface is arranged at the second end, and each pole piece extends from the contact surface at least partially in a radial direction to an end face, the radial direction being perpendicular to the axial direction, the end face is arranged around the cup-shaped recess, and at least one concentrated winding is arranged at each longitudinal leg, the at least one concentrated winding surrounding a respective longitudinal leg, for each of the plurality of coils, the longitudinal leg is made of a first material and the pole piece is made of a second material, the first material and the second material being different.

2. The magnetic levitation device according to claim 1, wherein for each of the plurality of coils, the longitudinal leg is made of sheet metal elements, and the elements are stacked in a circumferential direction of the stator.

3. The magnetic levitation device according to claim 1, wherein the first material is an electrical sheet metal.

4. The magnetic levitation device according to claim 1, wherein the second material is a powder composite material.

5. The magnetic levitation device according to claim 1, wherein for each of the plurality of coils, the contact surface is planar and is arranged at a surface of longitudinal leg which is perpendicular to the radial direction.

6. The magnetic levitation device according to claim 1, wherein for each of the plurality of coils, the contact surface is arranged at a surface of the longitudinal leg which stands perpendicular on the axial direction.

7. The magnetic levitation device according to claim 1, wherein for each of the plurality of coils, the contact surface is angled.

8. The magnetic levitation device according to claim 7, wherein for each of the plurality of coils, the contact surface has two partial surfaces, the first partial surface is perpendicular to the axial direction and the second partial surface is perpendicular to the radial direction.

9. The magnetic levitation device according to claim 1, wherein for each of the plurality of coils, the pole piece is angled, and the pole piece comprises two partial pieces, one partial piece extending in the radial direction and an other partial piece extending in the axial direction.

10. The magnetic levitation device according to claim 1, wherein each the coil core of the plurality of coil cores has a rounding at an axially upper end redirecting the coil core from the axial direction to the radial direction.

11. The magnetic levitation device according to claim 1, wherein for each of the plurality of coils, the end face of the pole piece is a curved surface.

12. The magnetic levitation device according to claim 10, wherein for each of the plurality of coils, the curved surface of the end face is coaxially with the cup-shaped recess.

13. The magnetic levitation device according to claim 1, wherein for each of the plurality of coils, the end face is wider with respect to the circumferential direction than a maximum extension of the contact surface in the circumferential direction.

14. The magnetic levitation device according to claim 1, wherein the rotor is capable of being driven magnetically without contact for rotation about the axial direction by a torque generated by the stator.

15. The magnetic levitation device according to claim 1, wherein the second material is a soft magnetic powder composite material.

16. An electromagnetic rotary drive which is a temple motor, the electromagnetic rotary drive comprising:
a magnetic levitation device according to claim 14;
the rotor with the disk-shaped or ring-shaped magnetically effective core, the rotor configured to be inserted into the cup-shaped recess, and is the rotor of the electromagnetic rotary drive.
